(19) United States
(12) Patent Application Publication
(10) Pub. No.: US 2008/0176811 A1
(43) Pub. Date: Jul. 24, 2008

(54) NOVEL FLAVONE GLYCOSIDE DERIVATIVES FOR USE IN COSMETICS, PHARMACEUTICALS AND NUTRITION

(76) Inventors: Bernadette Geers, Duesseldorf (DE); Ralf Otto, Bad Friedrichshall (DE); Albrecht Weiss, Langenfeld (DE); Dirk Petersohn, Koeln (DE); Klaus Rudolf Schroeder, Mettmann (DE); Kordula Schlotmann, Duesseldorf (DE)

Correspondence Address: COGNIS CORPORATION;PATENT DEPARTMENT, 300 BROOKSIDE AVENUE, AMBLER, PA, 19002, US

(21) Appl. No.: 12/057,425

(22) Filed: Mar. 28, 2008

Related U.S. Application Data
(63) Continuation of application No. 10/258,049, filed on Apr. 24, 2003, now abandoned.

(30) Foreign Application Priority Data
Apr. 18, 2000 (DE) 100 19 235.1

(51) Int. Cl. A61K 31/7048; C07H 17/06; C12P 19/60; A61P 17/00; A61Q 19/02

(52) U.S. Cl. 514/27; 536/8; 435/75

(57) ABSTRACT

Substituted and unsubstituted flavone or isoflavone glycoside derivatives of the formula [A¹-C(═O)O]ₘ—[X—O-Z]—[O—C(═O)-A²]ₙ, wherein [X—O-Z] represents a flavone or isoflavone glycoside structure, particularly a naringin residue, X is a flavone or isoflavone corresponding to formula (IIa) or formula (IIb):

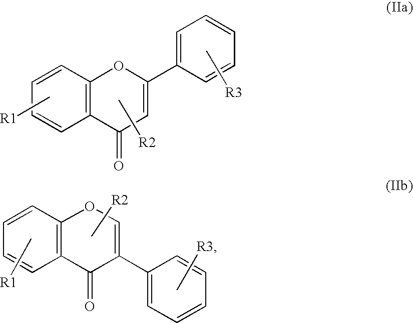

wherein the flavone or isoflavone residue is substituted one or more times and/or reduced one or more times; Z represents a mono-, di- or polysaccharide, which is acetally-bound at the benzopyran group to X and ester-substituted by —O—C(═O)-A²; [A¹-C(═O)] is an acyl group on the flavone or isoflavone; A¹ and A² independently, represent a polyunsaturated C₁₅₋₂₆ alkenyl group containing at least four isolated and/or at least two conjugated double bonds, or an arylaliphatic radical with 1-to-4 methylene groups between the ester group and the aromatic ring; [C(═O)A²] is an acyl group; n is an integer other than 0; m is an integer, including 0; and R1, R2 and R are hydroxyl groups or hydrogen atoms.

### CROSS-REFERENCE TO RELATED APPLICATIONS

This application is a continuing application of U.S. patent application Ser. No. 10/258,049, filed Apr. 24, 2003, which claimed priority under 35 U.S.C. §371 based upon International Application No. PCT/EP01/04151, having an International Filing Date of Apr. 11, 2001, the entire contents of each of which are herein incorporated by reference.

### BACKGROUND OF THE INVENTION

1. Field of the Invention

This invention relates to new biologically active flavone and isoflavone glycoside derivatives corresponding to general formula (I):

[A₁-C(═O)O]ₘ—[X—O-Z]-[O—C(═O)-A₂]ₙ (I)

of aliphatic and arylaliphatic carboxylic acids, to processes for their production, to cosmetic and/or pharmaceutical preparations containing these compounds and to their use as additives in human nutrition and animal feeds.

2. Background and Related Art

In the cosmetics field, the use of active substances is becoming increasingly more important. The active substances which have already been used in cosmetics have not always been natural substances. Much research work has been devoted to optimizing known active substances and to producing new active substances.

In the broadest sense, active substances are substances which—occurring or supplied in relatively small quantities—are able to develop strong physiological activity. Such substances would include hormones, vitamins, enzymes, trace elements, etc. and also pharmaceuticals (medicaments), feed additives, fertilizers and pesticides. Synergism is also observed in many cases.

### Flavones and Isoflavones/Flavonoids and Isoflavonoids or Flavone Glycosides And Isoflavone Glycosides

Flavones are 2-phenyl-4H-1-benzopyran-4-ones in which hydroxyl groups may be present or even missing at various positions of the rings. One example of a flavone is apigenin of which the chemical name is 2-(p-hydroxyphenyl)-4H-1-(5,7-dihydroxybenzopyran-4-one (see Römpp, Chemie-Lexikon, 9th Edition, Vol. 2, pp. 1373/4). As the example mentioned shows, the additional hydroxyl groups are located at the phenyl and/or the benzopyran ring. In other words, flavones in the context of the present invention are the hydrogenation, oxidation or substitution products of 2-phenyl-4H-1-benzopyran-4-one (hydrogenation may take place in the 2,3-position of the carbon skeleton; by substitution is meant the replacement of one or more hydrogen atoms by hydroxy or methoxy groups). Accordingly, this definition includes flavans, flavan-3-ols (catechols), flavan-3,4-diols (leucoanthocyanidines), flavones, flavonols and flavonones in the traditional sense. Besides apigenin, the flavones according to the invention include, for example, chrysin, galangin, fisetin, luteolin, camphor oil, quercetin, morin, robinetin, gossypetin, taxifolin, myricetin, rhamnetin, isorhamnetin, naringenin, eryodictyol, hesperetin, liquiritigenin, catechol and epicatechol.

By contrast, isoflavones in the context of the present invention are the hydrogenation, oxidation or substitution products of 3-phenyl-4H-1-benzopyran-4-one (hydrogenation may take place in the 2,3-position of the carbon skeleton; by substitution is meant the replacement of one or more hydrogen atoms by hydroxy or methoxy groups). The isoflavones according to the invention include, for example, daidzein, genistein, prunetin, biochanin, orobol, santal, pratensein, irigenin, glycitein, biochanin A and formononetin.

Flavones and flavone glycosides (flavanoids), such as asparatin, orientin (lutexin), cisorientin (lutonaretin), isoquercetin, rutin, naringin and those mentioned above, and also isoflavones and isoflavone glycosides (isoflavonoids) are known to be scavengers of oxygen radicals and inhibitors of skin proteases so that they are actively able to counteract aging of the skin and scar formation. By virtue of their coloring properties, some flavones, such as quercetin, are used as food colorants. At the same time, their ability to trap oxygen radicals also enables them to be used as antioxidants. Some flavonoids are inhibitors of aldose reductase which plays a key role in the formation of diabetes damage (vascular damage, grey star). Other flavonoids (such as hesperidin and rutin) are used therapeutically, more particularly as vasodilating capillary-active agents.

The derivatizations carried out in accordance with the invention achieve an improved effect and greater bioavailability, as previously shown with reference to the example of salicin derivatives.

Many naturally occurring alkyl and phenol glucosides show antiviral, antimicrobial and, in some instances, anti-inflammatory activity. In view of their polarity, however, their bioavailability is poor and their selectivity too low. For example, salicin (a glycosidic active substance from willow bark) is a nonsteroidal anti-inflammatory agent (NSAIA) which, after derivatization (esterifications), shows distinctly improved activity. Recently, researchers succeeded in synthesizing new arylaliphatic salicin esters, such as phenylacetoyl salicin and phenyl butyroyl salicin, the esterification taking place preferentially at the primary OH groups of the salicin (first at the sugar, then at the benzyl group) in the salicin. By virtue of the arylaliphatic group, mass transport to the point of action is improved and the selectivity of the effect is increased. Thus, in contrast to unmodified salicin, these derivatives preferentially inhibit prostaglandin synthase 2 (less danger of side effects) (Ralf T. Otto, Biotechnologische Herstellung und Charakterisierung neuer pharmazeutisch aktiver Glykolipide, Dissertation (1999) ISBN 3-86186-258-1).

### PUFAs and CLAs

In the field of nutrition, polyunsaturated fatty acids (PFAs) and conjugated linoleic acids (CLAs) belong to the group of essential fatty acids and also show a positive effect when used in the prophylaxis of arteriosclerosis. Pharmaceutical effects are also important; they are capable of developing anti-inflammatory activity (inhibition of prostaglandin and leucotriene synthesis) and also thrombolytic and hypotensive activity.

According to the invention, PUFA is defined as a polyunsaturated fatty acid containing 16 to 26 carbon atoms, the fatty acid containing at least four isolated and/or at least two conjugated double bonds. Examples of PUFAs are the twelve octadecadienoic acids isomeric to linoleic acid (cis, cis, 9,12-octadecadienoic acid) which occur in nature and which have conjugated double bonds at carbon atoms 9 and 11, 10 and 12 or 11 and 13.

These isomers of linoleic acid (for example cis, trans, 9,11-octadecadienoic acid, trans, cis, 10,12-octadecadienoic acid, cis, cis, 9,11-octadecadienoic acid, trans, cis, 9,11-octadecadienoic acid, trans, trans, 9,11-octadecadienoic acid, cis, cis, 10,12-octadecadienoic acid, cis, trans, 10,12-octadecadienoic acid, trans, trans, 10,12-octadecadienoic acid) can be conventionally prepared by chemical isomerization of linoleic acid, these reactions leading exclusively to CLA mixtures varying widely in composition (for example Edenor® UKD 6010, Henkel KGaA) in dependence upon the reaction conditions. By virtue of their conjugated double bonds, these isomeric octadecadienoic acids are also known as conjugated linoleic acids (CLAs).

Although numerous pharmacologically active substances which engage, for example, in the inflammation cascade have already been described in the literature, there is a still a need for more effective, low side effect active substances. There is also a need for active substances which are readily absorbed and penetrate quickly into the skin and which, in addition, should readily lend themselves to incorporation in pharmaceutical or cosmetic formulations.

There is also a particular interest in the discovery of active substances which can prevent the aging processes affecting human skin.

Human skin is the largest organ of the human body. It has a very complex structure and consists of a plurality of various cell types and forms the interface between the body and the environment. This fact clearly illustrates that the cells of the skin are particularly exposed to physical and chemical exogenous signals of the environment. Many of these exogenous noxae contribute to the aging of the skin. The macroscopic phenomena of aging skin are based on the one hand on intrinsic and chronological aging and, on the other hand, on extrinsic aging by environmental stress. The ability of living skin cells to react to their environment changes with time. Aging processes take place, leading to senescence and ultimately to cell death. The visible signs of aged skin should be interpreted as an integral of intrinsic and extrinsic aging (for example by sunlight), the results of extrinsic aging accumulating in the skin over a prolonged period.

Exogenous signals are received by cells and lead to changes in the gene expression pattern, in some cases through complex signal transduction cascades. In this way, each cell reacts to signals from its environment with adaptation of its metabolism. For example, the cells of the skin notice the high-energy radiation of the sun and react to it by reversing their RNA and protein synthesis capacities. After a stress stimulus (for example sunlight), some molecules are increasingly synthesized (for example collagenase MMP-1) while others are produced to a lesser extent (for example collagen α₁). In addition, in many of the synthesis processes, no significant change will occur (for example TIMP-1). The induction of collagenase MMP-1 by sunlight or other stress factors is regarded as the main cause of the process of extrinsic skin aging. Collagenase MMP-1 destroys the most important constituent of the connective tissue of the skin, collagen, and thus leads inter alia to a reduction in the elasticity of the skin and to the formation of deep wrinkles. In young and unstressed skin, the activity of collagenase is regulated by a naturally occurring inhibitor TIMP-1 (Tissue Inhibitor of Matrix Metalloprotease-1). There is an extremely delicate balance between MMP-1 and TIMP-1 which is critically disturbed by exogenous stress. The expression of MMP-1 is intensified by skin stress such as, for example, exposure to sunlight. By contrast, the synthesis of the inhibitor TIMP-1 is not significantly affected. Accordingly, the effect of exogenous stress, such as sunlight for example, on the skin leads to excessive degradation of collagen. The result is premature ageing of the skin.

Efforts at cosmetically treating the effects of stress-induced aging of the skin have targeted the reduction of MMP-1 activity or the increased synthesis of collagen. The use of retinic acid or retinol is said to reduce the synthesis of MMP-1 in the skin or to increase the synthesis of collagen. However, the use of retinic acid for cosmetics is not permitted in Europe because of teratogenic properties. Cytotoxic effects, inadequate stability in formulations, unwanted side effects or even problematical natural colors limit the cosmetic use of such active substances as, for example, α-tocopherol, propyl gallate or various plant extracts.

Accordingly, the problem addressed by the present invention was to provide low side effect, highly effective substances which would be easy to process and to apply.

Flavone and isoflavone glycosides are known, for example, from nature. By contrast, esters of flavone or isoflavone glycosides where at least one of the hydroxyl groups of the sugar is esterified with an (unsaturated) carboxylic or fatty acid and where, in addition, another ester group is present between one of the hydroxyl groups of the flavone or isoflavone component and another unsaturated fatty acid are not known (either from plants, microorganisms or animal cells or synthetically produced).

### BRIEF SUMMARY OF THE INVENTION

It has surprisingly been found that certain flavone and isoflavone glycoside esters have improved biological availability, an improved effect and/or a broader action spectrum by comparison with the known individual components (fatty acid or (iso)flavone glycoside). In these (iso)flavone glycoside derivatives, the flavones or isoflavones are glycosidically linked to at least one sugar via at least one hydroxyl group. The sugar may be linked to the (iso)flavone residue through an OH group at the benzopyran ring or through an OH group at the phenyl ring of the (iso)flavone. The [A₁-C(═O)] group may also be linked to the (iso)flavone through an OH group at the benzopyran ring or through an OH group at the phenyl ring of the (iso)flavone residue. Preferably, the sugar is linked to the (iso)flavone residue through its benzopyran ring while the fatty/carboxylic acid is also linked to the (iso)flavone residue through its benzopyran ring or through its phenyl ring.

Suitable sugars are mono- and oligosaccharides, more particularly D-glucose, D-galactose, D-xylose, D-apiose, L-rhamnose, L-arabinose and rutinose. Examples of the flavone glycosides in the compounds according to the invention are rutin, hesperidin and naringin. Preferred examples of the isoflavone glycosides in the compounds according to the invention are daidzin and genistin.

### DETAILED DESCRIPTION OF THE INVENTION

The problem stated in the foregoing has been solved by the provision of the compounds according to the present invention.

The compounds according to the present invention are flavone and isoflavone glycoside derivatives corresponding to general formula (I):

[A₁-C(═O)O]ₘ—[X—O-Z]-[O—C(═O)-A₂]ₙ (I),

in which [X—O-Z] represents a flavone or isoflavone glycoside structure,
X is a flavone or isoflavone parent substance corresponding to formula (IIa) or (IIb):

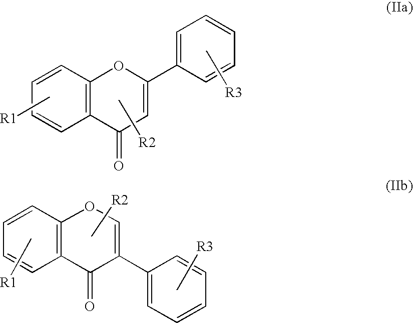

the (iso)flavone parent substance being substituted one or more times and/or reduced (hydrogenated) one or more times,
Z (sugar) represents a mono-, di- or polysaccharide which is acetally bound to X and substituted ester-fashion n-times by A₂,
[A₁-C(═O)] is an acyl group at the flavone or isoflavone parent substance,
A₁ and A₂ independently of one another represent a polyunsaturated C₁₅₋₂₅ alkenyl group containing at least four isolated and/or at least two conjugated double bonds or an arylaliphatic radical with 1 to 4 methylene groups between the ester group and the aromatic ring,
[C(═O)A₂] is an acyl group at the sugar Z,
n is an integer (1, 2, 3 . . . ), but not 0,
m is an integer (1, 2, 3, . . . ), including 0, and
R1, R2 and R3 are hydroxyl groups or hydrogen atoms.

Preferred sugars Z are generally monosaccharides. The following monosaccharides are particularly preferred: rhamnose, threose, erythrose, arabinose, lyxose, ribose, xylose, allose, altrose, galactose, glucose, gulose, idose, mannose, talose and fructose, the naturally occurring stereoisomers of the sugars being the preferred form. Other preferred sugars are disaccharides made up of the above-mentioned monosaccharides, the naturally occurring stereoisomers of the sugars again being the preferred form.

In a preferred embodiment, the (iso)flavone parent substance is linked to the sugar via a primary alcohol group of the sugar (for example via OH at C6 of the glucose). In another preferred embodiment, Z-O—X is the naringin skeleton corresponding to formula (III):

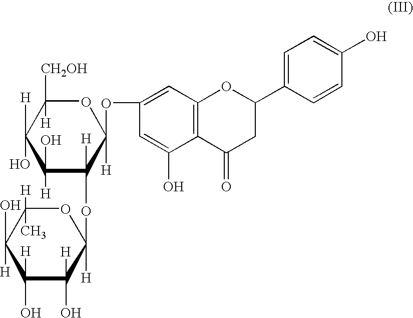

Other preferred flavones/flavonoids (X or X—O-Z) in general formula (I) are asparatin, orientin (lutexin), cisorientin (lutonaretin), isoqueroetin, naringin, rutin, camphor oil and quercetin.

Preferred compounds corresponding to general formula (I) are above all those where X—O-Z is naringin corresponding to formula (III) and As represents the acyl groups of the following acids: p-chlorophenylacetic, hydrocinnamic, stearic, 12-hydroxystearic, palmitic, lauric, oleic, coumaric, capric, cinnamic, 4-phenylbutyric, 4-hydroxyphenylacetic, 5-phenylvaleric acid or the mixtures commercially available as Edenor® UKD 6010 and UKD 7505. Edenor® UKD 6010 and UKD 7505, p-chlorophenylacetic and hydrocinnamic acid are particularly preferred acids. For all these combinations of naringin and the fatty acids mentioned, it is particularly preferred if n=1 or n=2 and, at the same time, m=0. Where n=1 (and m=0), the preferred position of A₂ is the primary OH group at the sugar in formula (III). However, all secondary OH groups of the sugar also represent preferred embodiments for the esterification. Where n=2 (and m=0), one esterification preferably takes place at the primary OH group and the second at one of the secondary OH groups of the sugar, more particularly at one of the two secondary OH groups at the same 6-membered ring or at one of the three secondary OH groups of the second 6-membered ring.

Other preferred compounds corresponding to general formula (I) are those where X—O-Z is naringin, A₂ represents the acyl groups of the following acids: p-chlorophenylacetic, hydrocinnamic, stearic, 12-hydroxystearic, palmitic, lauric, oleic, coumaric, capric, cinnamic, 4-phenylbutyric, 4-hydroxyphenylacetic, 5-phenylvaleric acid or the mixtures commercially available as Edenor® UKD 6010 and Edenor® UKD 7505; n=1 or n=2 and, at the same time, m=1. Where n and m are both 1, the preferred position of A₂ is the primary OH group in the sugar and that of A₁ is either the 5-OH group of the benzopyran ring or the 4′-hydroxy group of the phenyl ring. As in the case where m=0, however, A₂ can also be esterified through all the secondary OH groups of the sugar. Where n=2 and at the same time m=1, one esterification of A₂ takes place at the primary OH group and the second at one of the secondary OH groups of the sugar, more particularly at one of the two secondary OH groups at the same six-membered ring or at one of the three secondary OH groups of the second six-membered ring, and the esterification of A₁ takes place via the benzopyran ring or the phenyl ring.

It has surprisingly been found that the compounds corresponding to general formula (I) can be obtained by mild lipase-catalyzed esterifications.

Accordingly, the present invention also relates to a process for the production of the compounds of formula (I) according to the invention. The process according to the invention is characterized in that an acetal (from sugar and flavone/isoflavone parent substance) is esterified or transesterified with a polyunsaturated fatty acid (containing at least four isolated double bonds or at least two conjugated double bonds), such as a conjugated linoleic acid (octadecadienoic acid), with an arylaliphatic carboxylic acid, with an ester of these carboxylic acids or with an activated fatty acid derivative in the presence of one or more enzymes as catalysts. The esterification at primary OH groups of the sugar is preferred although secondary alcohol groups of the sugar can also be esterified.

Suitable enzymatic catalysts for the esterification of the above mentioned acids and the hydroxyl-containing acetal components include the hydrolases, particularly the lipases (ester hydrolases), such as the lipases from Candida rugosa (formerly Candida cylindracea), Candida antarctica, Geotrichum candidum, Aspergillus niger, Penicillum roqueforti, Rhizopus arrhizus and Mucor miehei.

A preferred lipase is the lipase (isoenzyme B) from Candida antarctica for which there are two reasons. Firstly, it shows particularly high selectivity in the esterification of the acetals with the unsaturated fatty acids although these are not among its typical substrates. Secondly, it does not show any interfacial activation (a key feature for the classification of hydrolases in the lipase group) because it lacks an important lipase structural feature, namely a mobile peptide chain at the active center (so-called lid).

In the production of the compounds according to the invention by the standard methods of chemical synthesis, mixtures of mono and poly-unsaturated products are generally formed through the presence of several free hydroxyl groups of the sugar and/or flavone/isoflavone parent substance, so that protective groups have to be introduced and removed if a certain compound is to be selectively synthesized.

However, selective esterification is crucial to the biological availability and compatibility of the substances according to the invention. Chemical synthesis leads to coarse product mixtures through inadequate regioselectivity. Accordingly, the enzymatic (see Examples), mild and regioselective synthesis described herein is of advantage. According to the invention, regiospecific means that only a certain OH group of a polyol is esterified. Accordingly, regioselective means that a certain OH group of a polyol is preferably but not exclusively esterified.

Once the compounds of formula (I) according to the invention have been produced by the process according to the invention, another process step generally has to follow in order to purify the required compound(s). Accordingly, another problem addressed by the present invention was to provide a process for purifying the compounds corresponding to formula (I) which is characterized in that it is a water-based two-phase extraction process using organic solvents by which the target compound can be selectively separated from the unreacted fatty acids. The organic solvent is preferably n-hexane, cyclohexane, THF, diethylether. Alternatively, purification can also be carried out by a chromatographic process on silica gel, preferably using ethyl acetate/methanol or dichloromethane/methanol mixtures with small contents of acetic acid and/or water, which may even be carried out in addition to a water-based two-phase extraction process with organic solvents.

Since the flavone/isoflavone glycosides of formula (I) according to the invention have good biological availability and activity, they may be used in cosmetic and pharmaceutical preparations and/or as food additives with the result that the quality of these very products is distinctly improved.

The compounds of formula (I) according to the invention have an inhibiting effect on skin proteases (anti-aging, anti-wrinkling), an antioxidative potential, a skin-lightening effect and a transcription-inhibiting effect. Particularly surprising is the skin-lightening effect (due to tyrosinase inhibition) of these compounds, especially the good skin-lightening effect of the compounds according to the invention in which Z-O—X is naringin and of which the primary OH group is esterified with phenylpropionic acid, hydroxyphenylacetic acid or p-chlorophenylacetic acid.

It has also been found that the compounds of formula (I) according to the invention, particularly those in which Z-O—X is naringin and of which the primary OH group is esterified with phenylpropionic acid, hydroxyphenylacetic acid or p-chlorophenylacetic acid, are capable of influencing the sunlight-induced expression of MMP-1, TIMP and Colα₁ in a cosmetically desirable manner and of thus counteracting the loss of collagen in the dermis. These compounds are therefore eminently suitable for cosmetic treatment of the skin to prevent sunlight-induced aging of the skin and/or to reduce its consequences.

The formation of collagen is influenced in particular by the extent of the expression of MMP and TIMP. The following strategies are possible for analyzing the factors involved in the process of homeostasis of skin cells exposed to sunlight:

### a) MMP:

quantification of the enzyme activity of MMP-1.
quantification of the synthetic MMP-1 protein.
quantification of the synthetic MMP-1-mRNA.

### b) TIMP:

quantification of the synthetic TIMP protein.
quantification of the synthetic TIMP-mRNA.

### c) Collagen:

quantification of the synthetic collagen protein
quantification of the synthetic Colα₁-mRNA.

The production of mRNA is the first and hence the most important step in the synthesis of proteins. Accordingly, active substances which have an effect on mRNA production automatically have an effect on the quantity of proteins and on enzyme activity. In a subsequent step, the outcome of the effects on mRNA production can be determined by detection of the protein collagen in the skin model itself.

It has been possible in accordance with the invention to show that naringin derivatives according to the invention are capable of reducing the expression of MMP, increasing the expression of TIMP, increasing the expression of Colα₁ and increasing the formation of collagen.

Although, in “photoaged” skin, MMP-1 is predominantly produced by fibroblasts, the reaction of the skin to stress may not be regarded as reactions of individual isolated skin cells. Instead, each cell is tied into a complex communication network. This network is responsible for the exchange of information between directly adjacent cells and also between localized cells situated further apart from one another such as, for example, the cells of the epidermis and the dermis. Signal molecules such as, for example, interleucines, growth factors (for example KGF, EGF and FGF), etc. are involved in the communication mechanisms between the cells of the skin. For this reason, analysis of the active-substance effects was carried out on skin models consisting of a dermal and an epidermal compartment.

It has also been found that the compounds according to the invention are considerably less phototoxic than conventional active substances against photoaging of the skin.

In addition, the compounds according to the invention lend themselves particularly readily to incorporation in lipophilic basic formulations and may readily be formulated as stable emulsions.

Accordingly, the compounds of formula (I) according to the invention are used for the production of cosmetic and/or pharmaceutical preparations and/or foods or animal feeds. The compounds according to the invention may be present or used in the form of the pure substance or as a mixture of plant extracts of various origins.

The (iso)flavones and their glycosides are preferably used as constituents of a mixture of substances obtained from a plant, more particularly a plant extract, in the preparations/additives. Plant-based mixtures such as these may be obtained in known manner, for example by squeezing out or extraction from such plants as citrus fruits (rutaceae family) or acacias.

Accordingly, the present invention also relates to the use of compounds corresponding to formula (I) for the production of cosmetic and/or pharmaceutical preparations; to their use as food supplements or additives in food preparations and in animal feeds; and to cosmetic and pharmaceutical preparations and foods/food preparations and animal feeds which contain (a) compound(s) corresponding to formula (I).

The cosmetic preparations obtainable using the compounds (I) in accordance with the invention, such as hair shampoos, hair lotions, foam baths, shower baths, creams, gels, lotions, alcohol water/alcohol solutions, emulsions, wax/fatty compounds, stick preparations, powders or ointments, may also contain mild surfactants, oil components, emulsifiers, superfatting agents, pearlizing waxes, consistency factors, thickeners, polymers, silicone compounds, fats, waxes, stabilizers, biogenic agents, deodorants, anti-dandruff agents, film formers, swelling agents, UV protection factors, antioxidants, hydroptropes, preservatives, insect repellents, self-tanning agents, solubilizers, perfume oils, dyes, germ inhibitors and the like as auxiliaries and additives.

The quantity in which the compounds according to the invention are used in the cosmetic (or even pharmaceutical) preparations is normally in the range from 0.01 to 5% by weight and preferably in the range from 0.1 to 1% by weight, based on the total weight of the preparations.

To produce pharmaceutical or even cosmetic preparations, the compounds of general formula (I) according to the invention—optionally in combination with other active substances—may be incorporated in typical galenic preparations, such as tablets, dragées, capsules, powders, suspensions, drops, ampoules, juices or suppositories, together with one or more typical inert carriers and/or diluents, for example corn starch, lactose, cane sugar, microcrystalline cellulose, magnesium stearate, polyvinyl pyrrolidone, citric acid, tartaric acid, water, water/ethanol, water/glycerol, water/sorbitol, water/polyethylene glycol, propylene glycol, carboxymethyl cellulose or fat-containing substances, such as hard fat or suitable mixtures thereof.

The daily dose required to obtain a corresponding effect in pharmaceutical applications is preferably 0.1 to 10 mg/kg body weight and more particularly 0.5 to 2 mg/kg body weight.

The food supplements and additives, such as sports drinks, obtainable using the compounds of formula (I) in accordance with the invention suitably contain the compound(s) of formula (I) in a quantity which, for a typical liquid intake of 1 to 5 liters per day, leads to a dose of these compounds of 0.1 to 10 mg and preferably 0.5 to 5 mg per kg body weight. One example of the use of the compounds of formula (I) in the food industry is their use as colorants and/or seasonings.

The following are intended to exemplify the instant invention and are not intended to, and should not be interpreted as in any way limiting it.

### EXAMPLES

### Example 1

### Preparation of 6-O-cis-9,trans-11-octadecadienoyl naringin

2 g of D-(−)-naringin, 5 g of CLA (Edenor® UKD 6010), 12 g of molecular sieve, 15 ml of t-butanol and 10 g of immobilized lipase B from Candida antarctica were incubated for 40 hours with stirring (magnetic stirrer, 100 r.p.m.) at 60° C. in a 250 ml Erlenmeyer flask. The reaction was monitored by thin-layer chromatography (silica gel KG60 plates with fluorescence indicator; mobile solvent: ethyl acetate/methanol 10:1 v/v; visualization: UV detection and with acetic acid/sulfuric acid/anisaldehyde (100:2:1 v/v/v) immersion reagent. The product was extracted with 20 ml of n-hexane and purified by column chromatography (silica gel F60; mobile solvent: ethyl acetate/methanol 10:1 v/v). R_{f} value: 0.47 (ethyl acetate/methanol 10:1).

### Example 2

### Preparation of 6-O-naringin-(3-phenylpropionic acid)-ester

5.8 g of naringin, 1.5 g of 3-phenylpropionic acid, 3.7 g of molecular sieve, 15 ml of t-butanol and 11 g of immobilized lipase B from Candida antarctica were incubated for 24 hours at 60° C./100 r.p.m. in a 250 ml flask. The reaction was monitored by thin-layer chromatography (silica gel 60 F₂₅₄; mobile solvent: ethyl acetate/methanol 10:1 v/v; visualization by UV detection). On termination of the reaction, the conversion based on naringin amounted to 20%. The product was extracted with 20 ml of n-hexane and purified by column chromatography (silica gel F60; mobile solvent: ethyl acetate/methanol 10:1 v/v). R_{f} value: 0.16 (ethyl acetate/methanol 10:1 v/v). Yield: 0.85 g.

The column chromatographic separation was not optimized. Besides fractions containing the pure product, mixed fractions containing unreacted naringin were obtained. Only those fractions from the column chromatography which contained only the required product were used to determine the yield indicated.

### Example 3

### Preparation of 6-O-naringin-(p-Cl-phenylacetic acid)-ester

5.8 g of naringin, 1.7 g of p-chlorophenylacetic acid, 3.8 g of molecular sieve, 15 ml of t-butanol and 11 g of immobilized lipase B from Candida antarctica were incubated for 24 hours at 60° C./100 r.p.m. in a 250 ml flask. The reaction was monitored by thin-layer chromatography (silica gel 60 F₂₅₄; mobile solvent: ethyl acetate/methanol 10:1 v/v; visualization by UV detection). On termination of the reaction, the conversion based on naringin amounted to 20%. The product was extracted with 20 ml of n-hexane and purified by column chromatography (silica gel F60; mobile solvent: ethyl acetate/methanol 10:1 v/v). R_{f} value: 0.20 (ethyl acetate/methanol 10:1 v/v). Yield: 0.50 g.

The column chromatographic separation was not optimized. Besides fractions containing the pure product, mixed fractions containing unreacted naringin were obtained. Only those fractions from the column chromatography which contained only the required product were used to determine the yield indicated.

### Example 4

### Preparation of Other Naringin Derivatives

Naringin derivatives prepared as described in Example 1 (reaction with Novozym SP 435 for 48 h at 65° C., stirring speed 1200 r.p.m.). The reaction was monitored by thin-layer chromatography and the conversion (based on the naringin used) was determined.

| | | Conversion |
|---|---|---|
| 4.1 | Stearic acid | + |
| 4.2 | Palmitic acid | ++ |
| 4.3 | Lauric acid | ++ |
| 4.4 | Oleic acid | + |
| 4.5 | Coumaric acid | + |
| 4.6 | Capric acid | + |
| 4.7 | Cinnamic acid | + |
| 4.8 | 4-Hydroxyphenylacetic acid | ++ |
| 4.9 | 5-Phenylvaleric acid | ++ |
| 4.10 | 4-Phenylbutyric acid | ++ |
| 4.11 | 12-Hydroxystearic acid | + |
| 4.12 | Edenor ® UKD 6010 | + |

+ = up to 15% conversion
++ = over 15% conversion

### Example 5

### Inhibition of Tyrosinase Activity

Tyrosinases physiologially catalyze an important step in the synthesis of melanin (L-dopa to L-dopaquinone which is further cyclized and re-reacted by a tyrosinase to dopachromium). Accordingly, inhibition of the tyrosinase can lead to a skin lightening effect.

The activity of fungal tyrosinase (Sigma) was determined in the presence of various concentrations of the active substances according to the invention by enzymatic reaction of LDOPA to dopachromium. The absorption maximum of dopachromium (red-brown) is at λ=475 nm. The linear increase in the absorption (A) of the dopachromium per unit of time (t) is a measure of the activity of the tyrosinase (ΔN/Δt). The activity of the tyrosinase in the absence of the active substances (ΔA₁/Δt₁) was used as reference (100%). Under analogous conditions, the residual tyrosinase activity was determined in the presence of the active substances (ΔA₂/Δt₂). Each measurement was carried out twice in parallel runs. The variation of the results of the method is ca.±10%.

### Chemicals Used:

| | |
|---|---|
| L-3,4-dihydroxyphenylalanine (L-DOPA) | (Sigma) |
| KH₂PO₄ | (J. T. Baker) |
| Tyrosinase, 50,000 units | (Sigma) |
| KOH | |

### Solutions Required:

50 mM KH₂PO₄ buffer in bidist. water (adjustment to pH 6.5 with 1 M aqueous KOH)
2.5 mM L-DOPA in bidist. water
340 U/ml tyrosinase stock solution in cold KH₂PO₄ buffer, pH 6.5.
Stock solutions of the active substance to be tested in bidist. water or ethanol in which the concentration of the active substance was 10 times higher than indicated in the line “active substance concentration in the test system” under “results”.

### Reaction Cocktail:

10 ml KH₂PO₄ buffer

### 10 ml L-DOPA

9 ml bidist. water

Like the tyrosinase stock solution, the reaction cocktail was prepared just before the beginning of the test. The tyrosinase stock solution has to be kept in a refrigerator. The L-DOPA solutions should be stored in darkness and in tightly closed containers in the absence of oxygen. If it turns grey in color (oxidation by atmospheric oxygen), the solution must be freshly prepared.

### Test System (Sample Volume 1 ml) and Reaction Procedure:

33 μl tyrosinase stock solution
100 μl active substance stock solution
reaction cocktail to 1000 μl

The activity of the tyrosinase in the absence of the active substances was used as reference (100%). All samples were thoroughly mixed in a Vibrofix before the beginning of the measurement. The pH value was monitored and if necessary was adjusted to pH 6.5. The measurement was carried out with a Kontron Uvikon 860 photometer. The absorption of the dopachromium was detected for 5 mins. at 25° C. at the absorption maximum X of 475 nm, the measuring time being 20-30 s.

### Results:

Active substance: 6-O-naringin-(3-phenylpropionic acid)-ester from Example 2
ACTIVE substance concentration in the test system:

| | | |
|---|---|---|
| 0.005% | 0.05% | 0.5% (w/v in bidist H₂O) |

Residual tyrosinase activity in % (IC 50=0.18%)

| | | |
|---|---|---|
| 98.9 | 69.1 | 0.7 |

Active substance: 6-O-naringin-(p-Cl-phenylacetic acid)ester from Example 3
Active substance concentration in the test system:

| | |
|---|---|
| 0.01% | 0.1% (w/v in 98% ethanol) |

Residual tyrosinase activity in %

| | |
|---|---|
| 45.1 | 15.4 |

### Example 6

### Phototoxicity

Dermal fibroblasts of human skin were cultivated with increasing concentrations of retinol (Table 1), 6-O-naringin-(p-Cl-phenylacetic acid)ester (Table 2) and 6-O-naringin-(3-phenylpropionic acid)-ester (Table 3). The phototoxicity of the substances was measured by an MTT test. To determine phototoxicity, the treated cells were exposed to simulated sunlight corresponding to a dose of 10 J UV-A/cm². The vitality of untreated cells was put at 100% and all other values were related to that value.

The exposure of the cells was carried out with a sunlight simulator from the emission spectrum of which the UV-A component of the radiation was measured for quantification. The advantage of this experimental design is the fact that the complete spectrum of the sunlight is used so that the everyday situation is excellently simulated. By contrast, many other research laboratories use pure UV-A and for UV-B lamps.

TABLE 1
phototoxicity of retinol

| Retinol concentration (ppm) | Vitality (%; in brackets: SEM) |
|---|---|
| 0.0028 | 99 (7.4) |
| 0.014 | 95 (19.8) |
| 0.028 | 80 (25.9) |
| 0.14 | 28 (11.8) |
| 0.28 | 4 (2.1) |

TABLE 2
phototoxicity of 6-O-naringin-(p-Cl-phenylacetic acid)-ester

| Conc. of naringin derivative (ppm) | Vitality (% in brackets: SEM) |
|---|---|
| 5 | 115 (15) |
| 10 | 96 (17.2) |
| 50 | 81 (8.3) |
| 100 | 3 (1.7) |
| 500 | 3 (1.8) |

TABLE 3
phototoxicity of 6-O-naringin-(3-phenylpropionic acid)-ester

| Conc. of the naringin derivative (ppm) | Vitality (%; in brackets: SEM) |
|---|---|
| 5 | 125 (5.8) |
| 10 | 103 (19.8) |
| 50 | 98 (15.1) |
| 100 | 101 (8.3) |
| 500 | 29 (4.5) |
| 1000 | 2 (0.5) |

The results show that, compared with retinol, 6-O-naringin-(3-phenylpropionic acid)-ester and 6-O-naringin-(p-Cl-phenylacetic acid)-ester only show toxic effects in relatively high concentrations. Retinol is toxic in very low concentrations. The reduction in vitality by several powers of ten is proof of the strong phototoxicity of retinol.

### Example 7

### Effects on the Light-Induced Expression of MMP-1-, TIMP- and Colα₁-mRNA

The effects of 6-O-naringin-(3-phenylpropionic acid)-ester (Table 4) and 6-O-naringin-(p-Cl-phenylacetic acid)-ester (Table 5) on the light-induced expression of MMP-1, TIMP and Colα₁ were measured at subphototoxic concentrations. To this end, the quantity of mRNA was quantified for MMP1, TIMP and Colaα₁. Skin models were treated with the test substances for 12 hours and then exposed to simulated sunlight corresponding to a dose of 10 J UV-A/cm². After another 48 hours in the presence of the active substances, the RNA of the cells was prepared and analyzed by Northern blots with specific gene probes. To monitor the quantity of RNA used in the experiments, Northern blots were carried out with an 18S-specific gene probe. To quantify the signal intensities, the autoradiograms were evaluated by densitometry and the values of the signals for MMP1, TIMP and Colα₁, were related to the associated values of the 18S signals. The figures in Table 1 represent the densitometric quantification of the signals of a Northern blot after normalization thereof. The light-induced expression of MMP 1, TIMP and Colα₁ for untreated cells was put at 100% and all other values were related to that value.

TABLE 4
Effects of 6-O-naringin-(p-Cl-phenylacetic acid)-ester
on the expression of MMP 1, TIMP and collagen

| Conc. of naringin | | | |
|---|---|---|---|
| derivative (ppm) | MMP 1 | TIMP | Collagen |
| 0, unexposed | 100 | 100 | 100 |
| 0, exposed | 135 | 89 | 66 |
| 5, exposed | 129 | 62 | 56 |
| 50, exposed | 40 | 77 | 79 |

TABLE 5
Effects of 6-O-naringin-(3-phenylpropionic acid)-ester
on the expression of MMP 1, TIMP and collagen

| Conc. of naringin | | | |
|---|---|---|---|
| derivative (ppm) | MMP 1 | TIMP | Collagen |
| 0, unexposed | 100 | 100 | 100 |
| 0, exposed | 135 | 89 | 66 |
| 10, exposed | 167 | 104 | 74 |
| 100, exposed | 87 | 134 | 99 |

The exposure of skin models to simulated sunlight led to a strong induction of MMP 1-mRNA synthesis whereas the synthesis of collagen was down-regulated. The production of TIMP remained largely unaffected. Table 4 shows that 50 ppm of 6-O-naringin-(p-Cl-phenylacetic acid)-ester very effectively reduced the sunlight-induced expression of MMP-1. The expression of TIMP was only slightly affected, the expression of Colα₁ is distinctly increased in relation to the exposed, untreated sample. The treatment of the cells with 100 ppm of 6-O-naringin-(3-phenylpropionic acid)-ester reduced the sunlight-induced expression of MMP-1 to the level of the unexposed, untreated sample (Table 5). By contrast, the expression of TIMP increased by around 35%. The expression of Colαᵢ was increased to the level of the unexposed, untreated culture.

The percentage change in the expression of MMP, TIMP and Colα₁, in cultures of exposed fibroblasts after treatment with 50 and 100 ppm of the tested naringin derivatives by comparison with exposed, untreated cultures is shown in Table 6.

TABLE 6

| | 6-O-naringin-(p-Cl-phenyl- | 6-O-naringin-(3-phenyl- |
|---|---|---|
| Expression | acetic acid)-ester | propionic acid)-ester |
| of | (50 ppm) | (100 ppm) |
| MMP | −70% | −37% |
| TIMP | −15% | 50% |
| Colα₁ | 27% | 64% |

The concentrations shown in Table 6 led to a distinct inhibition of MMP expression and to increased Colα₁ production for both naringin derivatives. The 6-O-naringin-(3-phenylpropionic acid)-ester increased TIMP production considerably whereas 6-O-naringin-(p-Cl-phenylacetic acid)-ester had only a slight effect.

### Example 8

### Effect on Collagen Production

In order to demonstrate the increased production of collagen at protein level, fibroblasts were treated with the test substances for 5 days in a three-dimensional culture system. On the sixth day, the quantity of collagen formed compared with non-collagen protein was determined via the incorporation of titrated protein. Table 7 shows the percentage increase in the collagen component of the protein as a whole, as determined from treated fibroblast cultures against untreated cultures.

TABLE 7

| | 6-O-naringin-(3-phenyl- | | | 6-O-naringin-(p-Cl- | |
|---|---|---|---|---|---|
| | propionic acid)-ester | | | phenyl-acetic acid)-ester | |
| Conc. (ppm) | 1 | 10 | 100 | 5 | 50 |
| Increase in collagen | 8% | 8% | 19% | −3% | 41% |
| production | | | | | |

What is claimed is:

1-17. (canceled)

18. A composition comprising a compound of the formula:
[X—O-Z]-[O—C(═O)-A²]ₙ (1),
wherein [X—O-Z]- represents a residue of naringin, a flavone glycoside of the formula: 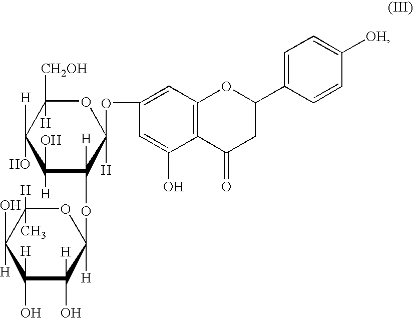
wherein the flavone glycoside is substituted one or more times; X is a flavone residue of formula (IIa), 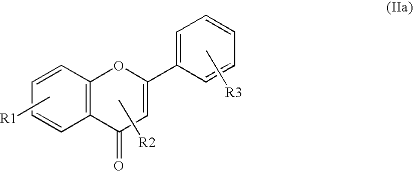
Z represents the residue of a saccharide which is acetally-bound to X at the benzopyran group and ester-substituted by —OC(═O)-A², wherein —C(═O)-A², independently, comprises at least one acyl group selected from the group consisting of residues of polyunsaturated C₁₆₋₂₆-fatty acids containing at least four isolated double bonds, residues of polyunsaturated C₁₆₋₂₆-fatty acids containing at least two conjugated double bonds, residues of arylaliphatic acids with 1-to-4 methylene groups between the —C(═O) group and the aromatic ring, a residue of capric acid, a residue of lauric acid, a residue of palmitic acid, a residue of stearic acid, a residue of oleic acid, a residue of 12-hydroxystearic acid, a residue of cinnamic acid and a residue of coumaric acid; n is 1 or 2; R1 and R2 are OH; and R3 is hydrogen.

19. The composition of claim 18, wherein in formula (I), —C(═O)A² comprises an acyl group from an acid independently selected from the group consisting of p-chlorophenylacetic acid, hydrocinnamic acid, cinnamic acid, 4-phenylbutyric acid, 4-hydroxyphenylacetic acid, 5-phenylvaleric acid, and mixtures of conjugated fatty acids, and n=.

20. The composition of claim 18, wherein in formula (I), n=1 and —C(═O)A² is attached to the primary OH group of a saccharide group in formula (III).

21. The composition of claim 18, wherein in formula (I), —C(═O)A² represents an acyl group from an acid independently selected from the group consisting of p-chlorophenylacetic acid, hydrocinnamic acid, cinnamic acid, 4 phenylbutyric acid, 4-hydroxyphenylacetic acid, 5-phenylvaleric acid, mixtures of conjugated fatty acids, and mixtures thereof; and n=2.

22. The composition of claim 18, wherein in formula (I), n=2 and one —C(═O)A₂ is attached to the primary OH group of the saccharide group in formula (III), and a second —C(═O)A₂ is attached to one of the secondary OH groups of the saccharide group in formula (III).

23. A process for making a compound of the formula:
[X—O-Z]-[O—C(═O)-A²]ₙ (I),
wherein [X—O-Z]- represents a naringin residue, X is a flavone or isoflavone residue of formula (IIa) or formula (IIb), respectively: 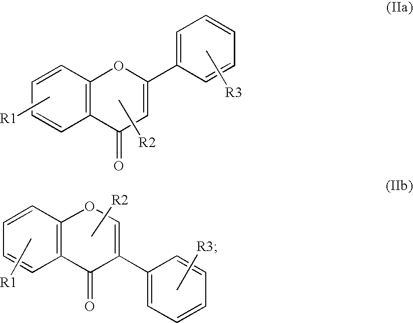
Z represents a saccharide which is acetally-bound to X at the benzopyran group and ester-substituted, by —OC(═O)A₂, wherein —C(═O)A₂, independently, comprises at least one acyl selected from the group consisting of residues of a polyunsaturated C₁₆₋₂₆-fatty acid containing at least four isolated double bonds, residues of a polyunsaturated C₁₆₋₂₆-fatty acid containing at least two conjugated double bonds, residues of arylaliphatic acids with 1 to 4 methylene groups between a —C(═O)— group and an aromatic ring, a residue of capric acid, a residue of lauric acid, a residue of palmitic acid, a residue of stearic acid, a residue of oleic acid, a residue of 12-hydroxystearic acid, a residue of cinnamic acid and a residue of coumaric acid; n is an integer of one or two; and R1 and R3 are OH; and R2 is hydrogen; comprising:
a. providing naringin;
b. providing a reactive component comprising at least one carboxylic acid or ester thereof selected from the group consisting of C₁₆₋₂₆-polyunsaturated fatty acids containing at least four isolated double bonds, C₁₆₋₂₆-polyunsaturated fatty acids containing at least two conjugated double bonds, arylaliphatic carboxylic acids having 1 to 4 methylene groups between a —C(═O) group and an aromatic ring, esters of arylaliphatic carboxylic acids having 1-to-4 methylene groups between a —C(═O) group and an aromatic ring, capric acid, lauric acid, palmitic acid, stearic acid, oleic acid, hydroxystearic acid, cinnamic acid, and coumaric acid;
c. providing at least one enzyme catalyst; and
d. esterifying or transesterifying (a) and (b) in the presence of (c) to form a compound of formula (I).

24. The process of claim 23, wherein the reactive component (b) comprises a conjugated linoleic acid.

25. The process of claim 23, wherein the enzyme catalyst (c) comprises at least one hydrolase.

26. The process of claim 25, wherein the hydrolase comprises a lipase produced by a microorganism selected from the group consisting of Candida rugosa, Candida antarctica, Geotrichum candidum, Aspergillus niger, Penicillium roqueforti, Rhizopus arrhizus and Mucor miehei.

27. The process of claim 23 further comprising purifying the compound of formula (I) by a water-based, two-phase extraction process with an organic solvent.

28. The process of claim 23 further comprising purifying the compound of formula (I) by means of a chromatographic purification process on silica gel.

29. A method for treating the aging process in human skin comprising contacting the skin with the composition of claim 18.

30. A method for lightening human skin comprising contacting the skin with the composition of claim 18.
